(19) United States
(12) Patent Application Publication
(10) Pub. No.: US 2026/0151208 A1
(43) Pub. Date: Jun. 4, 2026

(54) AUTONOMOUS AND REMOTE-CONTROLLED OPERATING LIGHT

(71) Applicant: ARCOL LLC, Houston, TX (US)

(72) Inventors: Connor GILLILAND, Tyler, TX (US); Anthony Christopher SALAZAR, El Paso, TX (US); Rawan ALJUBORY, Houston, TX (US); Carson Malcolm BENNER, West University Place, TX (US); Jack McQuaide WINGARD, Round Rock, TX (US); Zachary Bronson BUTTERFIELD, Frisco, TX (US)

(21) Appl. No.: 19/213,304

(22) Filed: May 20, 2025

Related U.S. Application Data
(60) Provisional application No. 63/727,527, filed on Dec. 3, 2024.

(51) Int. Cl. A61B 90/35; B25J 9/16; B25J 15/00; G05D 3/10; G06T 7/70

(52) U.S. Cl. CPC A61B 90/35; B25J 9/1676; B25J 15/0019; G05D 3/10; G06T 7/70; G06T 2207/30004; G06T 2207/30196

(57) ABSTRACT

The present disclosure generally provides a system for providing continuously optimized illumination by autonomously and/or remotely controlling an illumination source, such as a motorized ceiling-mounted arm with a light attached to its end. Autonomously and/or remotely controlling illumination is accomplished by tracking a user's area of focus through two cameras, autonomously positioning and/or repositioning the illumination source to follow the user's area of focus, autonomously positioning and/or repositioning to avoid casting shadows, and utilizing remote sensing technology, such as LiDAR technology, to avoid collisions during positioning and/or repositioning. The system can also be controlled by a remote operator and can toggle between autonomous and remote-controlled operation on command.

### CROSS-REFERENCE TO RELATED APPLICATIONS

This application claims benefit of U.S. Provisional Patent Application No. 63/727,527, filed Dec. 3, 2024, which is herein incorporated by reference in its entirety.

### BACKGROUND

### Field

Embodiments of the present disclosure generally relate to medical and surgical illumination systems and, more particularly, autonomous and/or remote controlled illumination systems, including autonomous and remote control with LiDAR (Light Detection and Ranging) collision avoidance.

### Description of the Related Art

Lighting systems serve as indispensable tools in medical facilities and play a vital role in the assessment and execution of various procedures in both hospital and clinical environments. Proper illumination is critical so that a medical provider such as a doctor, dentist, or veterinarian can clearly see the area of focus. This enables them to be more successful and efficient through any operation. Efficiency is also important because operating time is expensive; studies have shown an average cost of $46.04 per minute during surgical procedures.

Conventional lighting systems consist of a ceiling mounted pendant with one or more articulating arms having light heads on the ends thereof. These systems allow for flexibility of illumination from multiple angles, and the ceiling fixture improves mobility. However, these systems require manual intervention to be positioned and/or repositioned which can distract attendants and also introduce the potential for contamination of the sterile field and requires continuous sterilization. Studies have shown that these systems require repositioning every seven and a half minutes on average, causing a distraction in 64% of instances and requiring attendants to move in 10% of instances. This can be distracting, dissatisfying, and potentially dangerous for both the medical team and patients.

Current alternatives to ceiling mounted lighting systems exist, but they are not without limitations. Head-worn lights are common alternatives, but they can cause headaches, neck pain, and back pain after periods of extended use. Studies show that back, neck, and shoulder pain is found in 68.2%, 56.9%, and 46.2% of surgeons, respectively, and overall musculoskeletal pain in 87.2% of surgeons. Other alternatives such as an array of adjustable lights on the ceiling have been developed, but they remain largely unintegrated into medical care due to their expensive and unfamiliar nature.

Considering existing art, there is a need for improved surgical lighting systems to reduce the need for physical interaction and improve procedural efficiency.

### SUMMARY

The present disclosure generally provides a system for providing continuously optimized illumination by autonomously and/or remotely controlling an illumination source, such as a motorized ceiling-mounted arm with a light attached to its end. In one embodiment, autonomously and/or remotely controlling an illumination source is accomplished by tracking a user's area of focus through two cameras, autonomously positioning and/or repositioning the light to follow the user's area of focus, autonomously positioning and/or repositioning the light to avoid casting shadows, and utilizing LiDAR technology to avoid collisions during positioning and/or repositioning. The system can also be controlled by a remote operator and can toggle between autonomous and remote-controlled operation on command.

In some embodiments, a surgical lighting system includes an actuatable arm, a light positioned at a first end of the actuatable arm, a first camera coupled to the light, the first camera configured to detect a laser beam, an overhead camera positioned at a second end of the arm, the overhead camera configured to detect the laser beam, a wearable head unit comprising a laser for generating the laser beam, and a controller configured to receive data from the overhead camera and the first camera indicative of a position of the laser beam, and in response to the received data, actuate the actuatable arm to adjust the position of the light.

In some embodiments, an autonomous surgical lighting system includes an actuatable arm, a light source coupled to the actuatable arm and configured to generate a light, a plurality of cameras, and a controller, comprising a memory comprising executable instructions, and a processor in data communication with the memory and configured to execute the executable instructions to receive image data from the plurality of cameras, determine a location of a user's point of focus in a surgical field based on the image data, and actuate the actuatable arm to adjust a position of the light to the location.

In some embodiments, a system for lighting a surgical field includes a memory comprising executable instructions that when executed by a processor perform a method, comprising receiving information from a light head camera indicative of a user's point of focus, determining whether the user's point of focus is within a surgical field, and adjusting a light head to track the user's point of focus by adjusting one or more motorized joints along an actuatable arm until the user's point of focus is within an adjustable tolerance to the center of the image.

### BRIEF DESCRIPTION OF THE DRAWINGS

So that the way the features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIGS. 1A-1C are multiple views of an illumination system in a surgical suite, according to one or more embodiments.

FIG. 2 is a motorized light head subsystem, according to one or more embodiments.

FIG. 3 is a central rotary mount, according to one or more embodiments.

FIG. 4 is a schematic view of improved safety glasses for line-of-sight tracking, according to one or more embodiments.

FIGS. 5A-5B are computer views from an overhead camera and light head camera, according to one or more embodiments.

FIG. 6 illustrates a LiDAR map generating a 3D point cloud corresponding to objects in the operating environment, while monitoring fields for potential hazards, according to one or more embodiments.

FIG. 7 is a schematic view showing zones monitored by LiDAR scanner data, according to one or more embodiments.

FIG. 8 is a flow chart of commands and feedback in controller systems, according to one or more embodiments.

FIG. 9 is a flow chart of logic followed by a microprocessor, according to one or more embodiments.

FIG. 10 is a remote control, according to one or more embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure generally provides a system for providing continuously optimized illumination by autonomously and/or remotely controlling an illumination source, such as a motorized ceiling-mounted arm with a light attached to its end. Autonomously and/or remotely controlling illumination is accomplished by tracking a user's area of focus through two cameras, autonomously positioning and/or repositioning the illumination source to follow the user's area of focus, autonomously positioning and/or repositioning to avoid casting shadows, and utilizing remote sensing technology, such as LiDAR technology, to avoid collisions during positioning and/or repositioning. The system can also be controlled by a remote operator and can toggle between autonomous and remote-controlled operation on command.

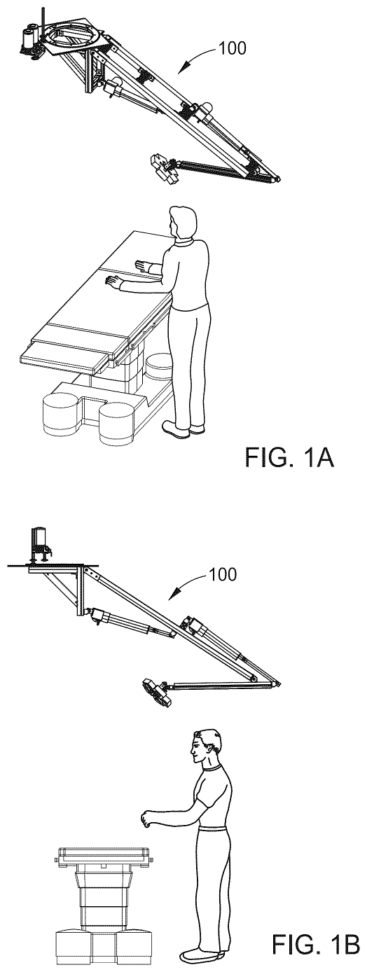

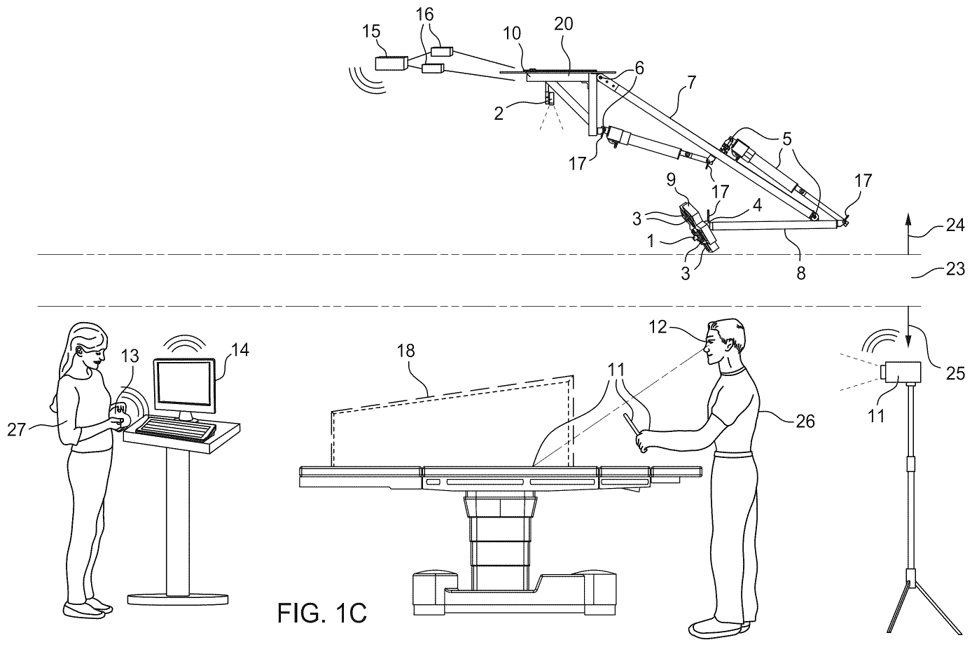

FIGS. 1A-1C are schematic views of a surgical suite utilizing an embodiment of the illumination system (100). FIGS. 1A-1C are described together herein for clarity and depict a surgical table and illumination system mounted in operable relationship to the surgical table. Embodiments contemplated herein provide an illumination system which provides autonomous and/or remote control of the illumination system. The illumination system includes a microprocessor (15) which, in both autonomous and remote-controlled operation, controls the illumination system. The illumination system is comprised of a central mount (10), two arms or booms (7, 8), a light head (9), and motorized joints between each (6, 5, 4, respectively). In one embodiment, these joints (4, 5, 6) may be linear actuators, while in other embodiments they may be stepper, servo, or other rotary motors. These motorized joints (4, 5, 6) may have one or multiple degrees of freedom in different embodiments. The microprocessor (15) communicates with motor controllers (16) to control the motorized joints (4, 5, 6) and motor feedback sensors (17) to monitor the position of the arms or booms and the motorized joints (4, 5, 6). The motor controllers (16) and motor feedback sensors (17) may be separate from or integrated into the microprocessor (15) and/or the motorized joints (4, 5, 6). The light head (9) may have multiple LED arrays (3) to provide illumination. These LED arrays (3) may have different functions of control such as brightness and/or adjustable focal length. The central mount (10) may feature a slip ring (20) to allow rotation of the illumination system 100 without causing torsional damage to the wiring. The light head (9) may be brightly colored, such as bright yellow or orange, to provide additional safety by further reducing the risk of a person in the operating area, such as the user (26), inadvertently colliding with the light head (9).

The light head (9) may also have one or more additional sensors thereon to prevent collision. For example, the light head (9) may include ultrasonic or other sensors, which provide redundancy and thus an additional layer of safety. For example, the additional sensors may sense items or obstructions in the event they are missed by the LiDAR, or may even be useful in the event of an earthquake.

During autonomous operation, the system tracks the user's (26) point of focus (19) using two cameras. The user's point of focus (19) may be indicated in various ways, including for example, colored glove(s), colored tool(s), or a pointer, such as a laser beam.

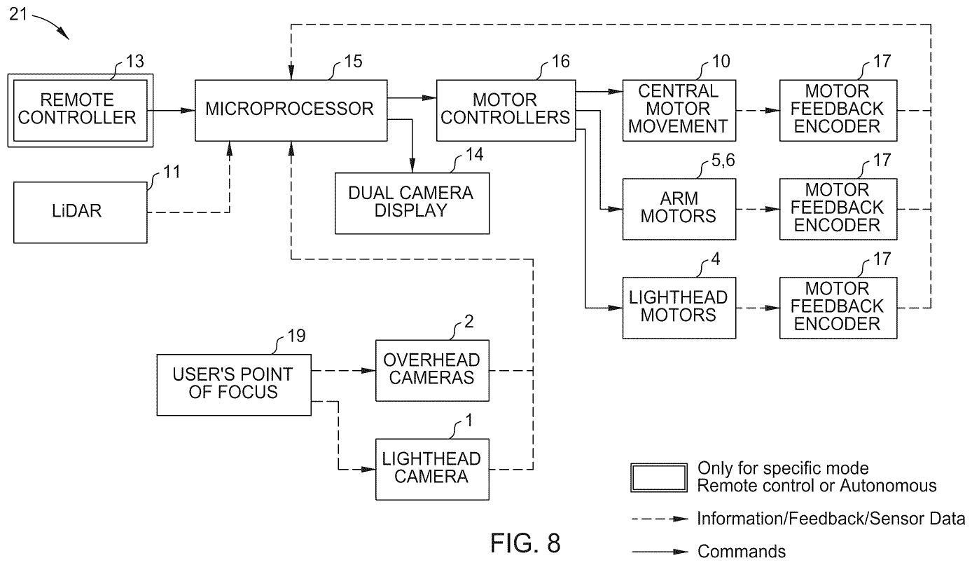

A first camera, such as a light head camera (1) is mounted on the light head (9) and a second camera, such as an overhead camera (2) mounted, for example, overhead on the central mount (10). The first camera (1) and the second camera (2) provide image data to the microprocessor (15) for analysis, as shown in the Info/Command Flow Chart (21) shown in FIG. 8. The light head (9) tracks the user's point of focus (19) to provide continuously optimized lighting while limiting unnecessary arm movements. The system is preferably limited to tracking within a defined surgical field (18) to prevent contact with attendants and other equipment within the room. This allows the user (26) to look away, such as to talk to another person in the room, without the light head (9) tracking and shining in an undesirable location, such as in someone's eyes.

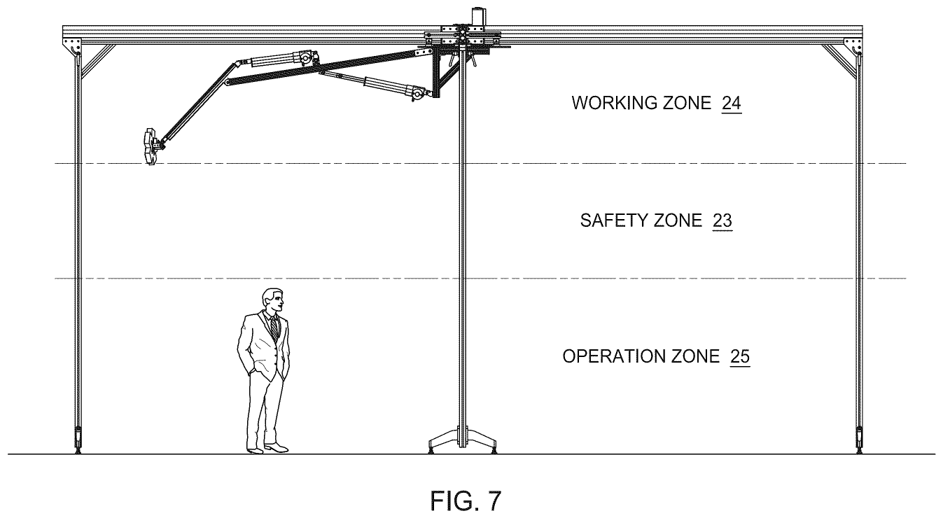

A LiDAR scanner (11) scans the room to monitor defined fields (28) and provides the microprocessor (15) with data to establish one or more zones. As shown by way of example in FIG. 7, the zones in one embodiment, consist of the operation zone (25) as established by the tallest object or person in the surgical suite and below, the working zone (24) as established by the lowest extension of the illumination system 100 and above, and the safety zone (23) which is maintained between the working zone (24) and operation zone (25) during system movement in positioning and/or repositioning. The safety zone (23) is meant to prevent collisions between the illumination system 100 and any objects and/or people in the room during positioning and/or repositioning. The working zone (24) may be defined through inverse kinematics, and/or LiDAR data. In another embodiment, the LiDAR scanner (11) may be mounted to the illumination system 100 to monitor the area(s) in its immediate vicinity, or the LiDAR scanner (11) may be mounted to the central mount to monitor the surgical suite from above. There may be one or multiple LiDAR scanners (11) used in any combination of the above or in other unique embodiments for collision avoidance and/or guidance. LiDAR scanner(s) (11) may be used to provide 3D point cloud data to the microprocessor (15) for use in position optimization.

During remote control operation, a remote operator (27) provides commands to the microprocessor (15) via a remote control (13) as shown in the embodiment of FIG. 10. A user interface (14) is used to provide the remote operator (27) with visual feedback during control. The user interface (14) may show image(s) from either or both the light head camera (1) and/or the overhead camera (2), as well as additional information such as the system's power usage or other key performance indicators. The user interface (14) may be customized to the user's preferences.

The microprocessor (15) may communicate with the LiDAR scanner (11), remote control (13), and user interface (14) through wired and/or wireless connection.

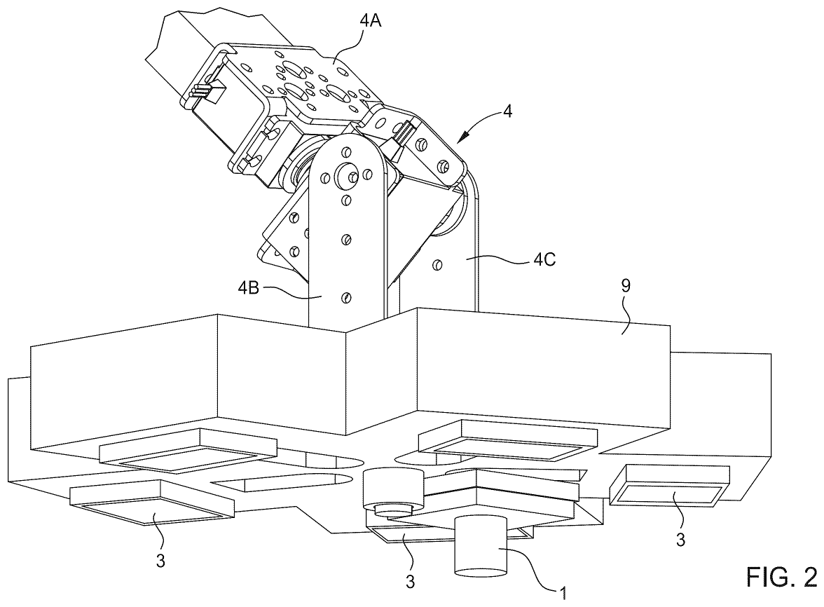

FIG. 2 is a perspective view of the light head subsystem. This subsystem consists of a camera (1), light emitting diode (LED) arrays (3), and a motorized joint (4), comprising subparts (4A-4C), which in some embodiments has two degrees of freedom. In other embodiments, the motorized joint (4) may have additional degrees of freedom as needed to provide the flexibility of controlling illumination. The LED arrays (3) provide illumination and can be designed and/or configured to provide any pattern and quality of illumination. The light head camera (1) is used to gather images for the microprocessor (15) used for tracking the user's (26) point of focus (19). The light head camera (1) also provides image data for the user interface (14). The light head (9) is mounted to the end of the actuatable arm (8) with at least two axes of freedom to allow for full hemispherical range of motion. In this embodiment, the joint (4) is motorized by two servo motors mounted to control the light head's (9) orientation in at least two axes. In other embodiments, existing light heads may be retrofitted with a camera and motors to provide this functionality. In further embodiments, the servos may be mounted to the arm on an existing system to motorize an existing light head's joint. In still further embodiments, servo motors may be replaced with linear actuators connected to the arm (8) and light head (9) at 90 degrees to each other to similarly control two or more axes of motion.

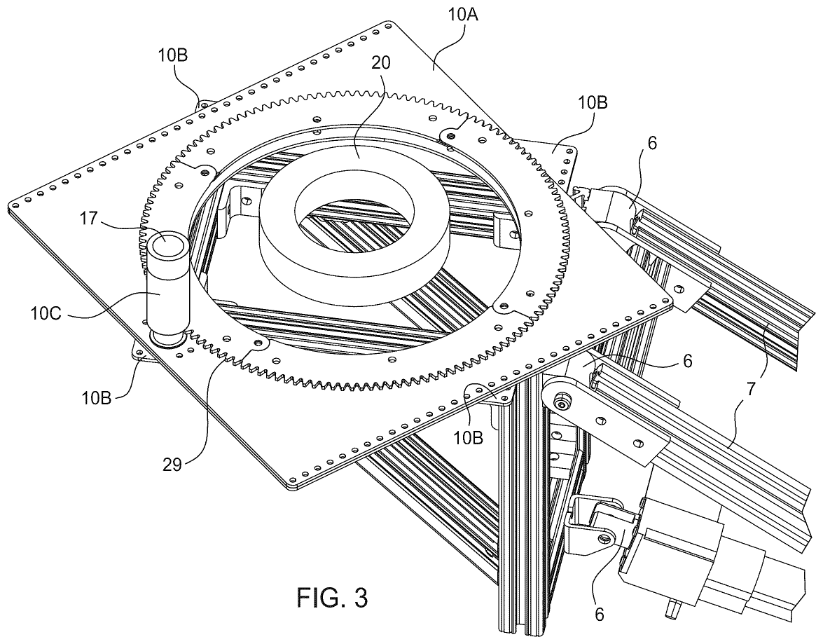

FIG. 3 is an enlarged view of the central mount (10) of the system. The central mount (10) has three parts: a stationary part (10A) that mounts the system to the ceiling, a rotating part (10B) attached to the arm, and the motor (10C) that controls the arm. The stationary part (10A) is fixed to the ceiling supports via one or more brackets. The rotating part (10B) is connected to the actuatable arms (7, 8) and rotates with the large driven gear (29) disposed above the rotating part (10B). The motor (10C) drives the rotating part (10B) to rotate the arm (7) about the central mount (10). In this embodiment, the motor (10C) is connected to an encoder (17) to accurately track the position of the arm. A slip ring (20) is built into the central mount (10) to allow wires to pass to the illumination system 100 without being subjected to twisting or torsional stress. In alternative embodiments, the central mount (10) could be belt or chain driven or could be modularly attached to control an existing lighting system.

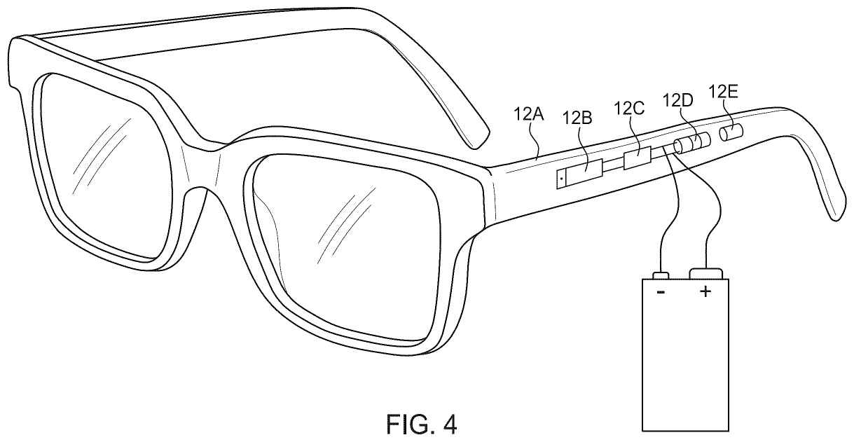

FIG. 4 is a perspective view of an improved pair of safety glasses (12) to be worn by a user, such as a surgeon in an operating environment. A laser module (12B) is mounted to the frame of the glasses (12A). The laser module (12B) may be adjustably mounted to the frame. In one embodiment, the laser module (12B) is mounted using a soft adhesive that is malleable so that the user (26) can adjust the position of the laser to align the laser with their point of focus (19). In another embodiment, the laser module (12B) may be mounted with a hard frame fitted with screws to make fine adjustments to the position of the laser module (12B). The laser module (12B) is connected to a small on/off switch (12C). The laser module (12B) may be powered via an external battery (12D) which may be mounted to the frame of the glasses (12A) or may be connected via wires with a clip to attach to the user's (26) clothing. In one embodiment, the laser module (12B) may be of a specific color. In another embodiment, the laser module (12B) may be of the infrared spectrum. A gyroscope (12E) may be mounted on the glasses frame (12A) to monitor the angle and direction of the user's (26) line of sight.

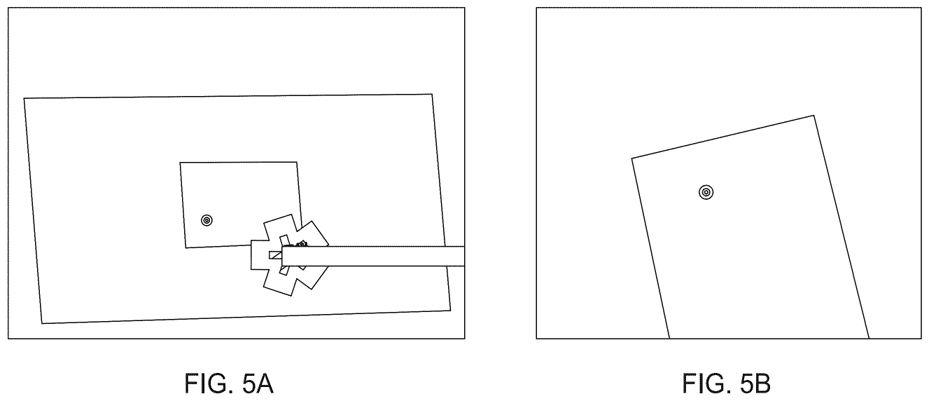

FIGS. 5A-5B depict the user interface (14) screen. This screen shows images from both cameras (1, 2) in real time. During remote control operation the images provide the remote operator (27) with feedback. During autonomous operation, the images are used by the microprocessor (15) to track the user's point of focus (19) and are presented with the point being tracked, highlighted, and marked. FIG. 5A depicts the screen showing an image from the overhead camera (2) and FIG. 5B depicts the screen showing an image from the light head camera (1). The image from the overhead camera (2), shown in FIG. 5A, captures a larger field of view, including the surgical field and the light head camera. The image from the light head camera (1), shown in FIG. 5B, captures a smaller field of view, focused solely on the surgical field, which is useful for the user for tracking purposes. FIGS. 5A and 5B show a dot, which represents the user's point of focus (19), represented by a laser point in this example, in the surgical field. In other embodiments, the user interface (14) could be customized to resize or reorient the images or to show additional data such as elapsed time, operating mode, light level, etc.

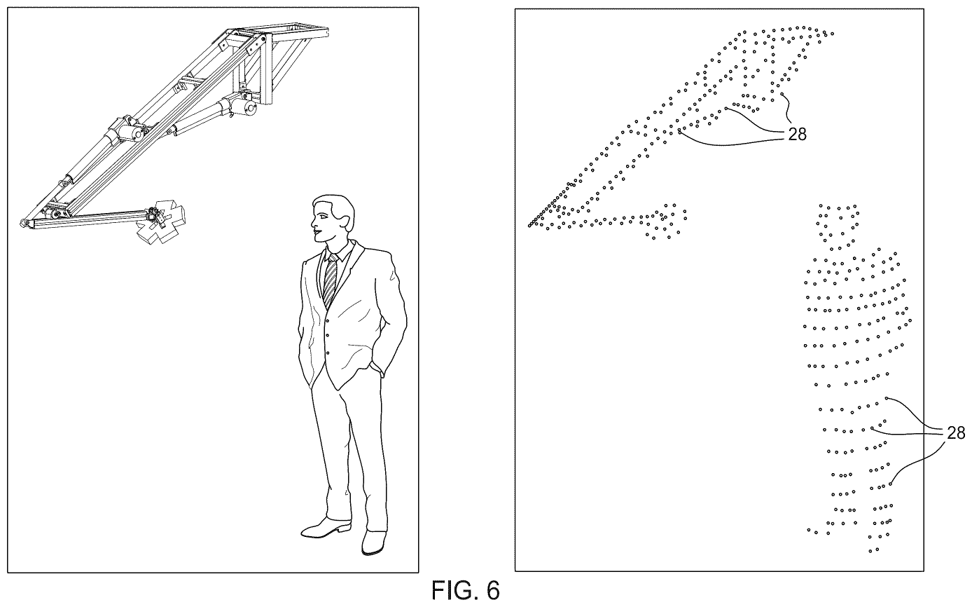

FIG. 6 depicts point cloud data from the LiDAR scanner (11) of the occupants and structure in the view field. A camera image of a person and the lighting system is shown on the left. The corresponding 3D point cloud data (28) produced by the LiDAR scanner (11) of the camera image is shown on the right. The LiDAR scanner (11) monitors fields (not shown). The fields are generally rectangular prisms; however, if the LiDAR scanner (11) is mounted overhead the fields may be conical or triangular prisms. In operation, when an object is detected within a field (28) based on the LiDAR map, the field turns yellow, and the LiDAR scanner (11) informs the microprocessor (15) that there is an object in that field. This allows the microprocessor (15) to know where the top of the tallest object is located, so that it can define the operating zone (25), which is shown in FIG. 7. In another embodiment, the microprocessor (15) may use other data, such as field data, 3D point cloud data, or inertial measurement unit (IMU) data, in addition to or separate from the field status produced by the LiDAR scanner (11). In another embodiment, the LiDAR scanner (11) may be mounted to the central mount (10) or to the illumination system 100 to monitor the surgical suite from above and/or the area(s) in the immediate vicinity of the physical system. In other embodiments, the LiDAR scanner (11) may be stationary or may rotate with the illumination system 100.

FIG. 7 is a schematic overlaid on the lighting system and its environment. It highlights the different zones set by the microprocessor (15) based on LiDAR data and inverse kinematics. The “Operating Zone” (25) is set by the tallest object in the area, in this case a person, to track the upper limit of objects in the area. The operating zone (25) can include an area of expected positioning of, or movement by, users such as surgeons and other operating staff, during the performance of a surgical procedure. The “Working Zone” (24) is set by LiDAR data and/or inverse kinematics to track the location of the physical lighting system. The working zone (24) can include an area within which the lighting system can freely actuate without interfering with objects or users in the environment. The “Safety Zone” (23) is the area between the operating zone (25) and the working zone (24) to prevent collisions between the lighting system and other objects during positioning and/or repositioning.

The Working Zone 24 is the area of least risk for a collision as it does not include objects that may cause an obstruction. The Safety Zone 23 is the area of medium risk for a collision as it might include objects that may cause an obstruction. The Operating Zone 25 is the area of greatest risk for a collision because it will include objects that may cause obstructions.

FIG. 8 is a flow chart of the information and commands (21) of the control system. The microprocessor (15), which is in data communication with a memory and storage, passes commands to the motor controllers (16) that move the motorized joints (4, 5, 6) of the system. The microprocessor (15) passes images and other data to the user interface display (14). The microprocessor (15) also receives inputs of either commands or information. If in remote control mode, the microprocessor (15) receives commands from the remote operator (27), for example using a remote control 13, and passes the commands to the motor controllers (16). If in autonomous mode, the microprocessor (15) receives information from the LiDAR scanner (11), first and second cameras (1, 2), and motor feedback sensors (17). The LiDAR information is analyzed and used for collision avoidance as described above. The cameras provide image data from both the light head camera (1) and the overhead camera (2). The motor feedback sensors (or encoders) (17) provide current positions of each motorized joint (4, 5, 6) and the central mount (10) which are analyzed with inverse kinematics to find the position of the lighting system and, if necessary, decide how to move the motors to reach a new desired position.

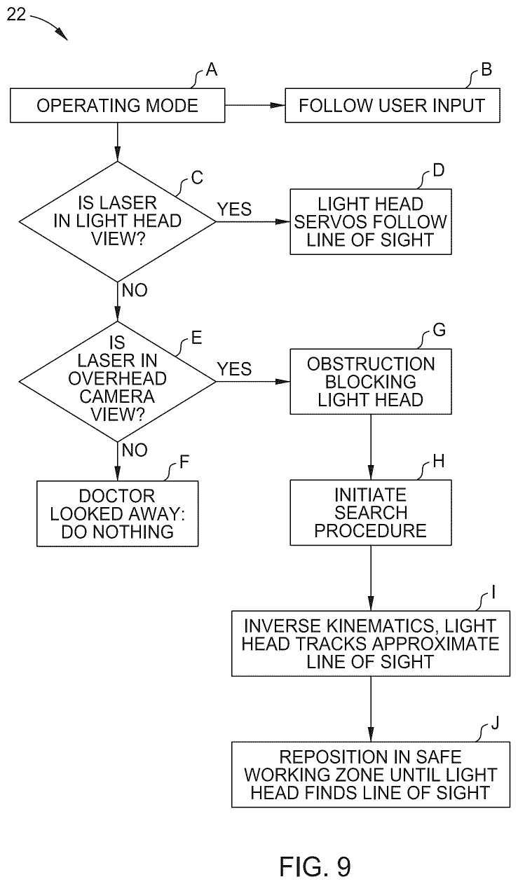

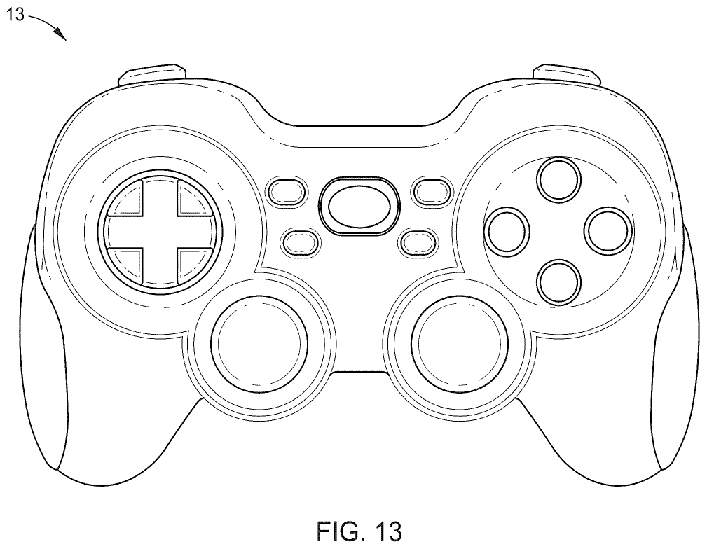

FIG. 9 is a flow chart (22) of the logic followed by the microprocessor (15). The logic flow chart is a generalization of the operations (A-J) constantly being executed by the microprocessor (15) and may be altered in other embodiments. At operation (A), the microprocessor (15) checks whether it is in autonomous or remote-control operating mode. If it is in remote-control mode, at operation (B) it simply follows commands from the remote operator. If microprocessor (15) is in autonomous mode, microprocessor (15) gathers information from all inputs for later analysis as necessary. At operation (C), microprocessor (15) analyzes the image data from the light head camera (1). If the light head camera (1) can see the user's point of focus (19), then at operation (D) the light head (9) will move to track it. This is done by adjusting the motorized joint (4) until the user's point of focus (19) is within an adjustable tolerance to the center of the image. If the light head camera (1) cannot see the user's point of focus (19), then at operation (E) the microprocessor (15) analyzes the overhead camera (2) image data. If the overhead camera (2) cannot see the user's point of focus (19) or it is out of the surgical field (18), then at operation (F) the system does nothing because the user's point of focus (19) is outside of the defined surgical field (18). If the overhead camera (2) can see the user's point of focus (19) within the defined surgical field (18), then at operation (G) the system will identify that the light head (9) must be blocked by some obstruction, so it will initiate a search procedure at operation (H).

This microprocessor (15) first uses inverse kinematics to estimate the location of the illumination system 100 and estimates where the user's point of focus (19) is. The microprocessor (15) then begins repositioning the illumination system 100 while directing the light head (9) towards the approximate user's point of focus (19) throughout the reposition. The microprocessor (15) uses the LiDAR fields (28) to ensure the safety zone (23) is maintained during the reposition. The system will search for another position from which the light head camera (1) can see the user's point of focus (19). Once this position is found, the system will resume tracking the user's point of focus (19) with the light head's motorized joint (4) at operation (D). At any point the remote operator (27) may change modes from autonomous (operations C-J) to remote controlled mode at operation (B) or back. The tolerance for how long the light head camera (1) must not see the user's point of focus (19) before initiating the search procedure may be adjusted to address noise in the system and prevent unnecessary repositions. In another embodiment, the system may include an analysis of LiDAR point cloud data in position optimization. In another embodiment, gyroscopic data from the user (12E, 26) may be used in position optimization, such as to align the light head (9) as close to the user's (26) line of sight.

FIG. 10 is an image of a remote control (13) that may be used to control the system while in remote-control mode. The remote operator (27) can toggle between autonomous and remote-control mode at any time. In alternative embodiments, the commands may be changed and customized to the remote operator's (27) preferences. In alternative embodiments, joysticks may also be utilized for controlling the system.

The foregoing description references laser and cameras as examples of ways to mark the user's point of focus (19). However, it is further contemplated that in other embodiments, the user's point of focus (19) may be marked using one or more gyroscopes, ultrasonic sensors, or other distance sensors. Using data about the direction of the user's point of focus (19) from the gyroscope and distance data from the sensor, the system could create a vector to define the user's point of focus (19) as a 3D point. Using LiDAR point cloud data or a distance sensor on the light head, the same targeting performance can be achieved with vector math rather than cameras.

Still further, the one or more gyroscopes, ultrasonic sensors, and distance sensors may be used in combination with the lasers and cameras for redundancy, resulting in decreased collisions and thus improved safety.

While the foregoing is directed to embodiments of the present disclosure, other and further embodiments of the disclosure may be devised without departing from the basic scope thereof, and the scope thereof is determined by the claims that follow.

What is claimed is:

1. A surgical lighting system, comprising:
an actuatable arm;
a light positioned at a first end of the actuatable arm;
a first camera coupled to the light, the first camera configured to detect a laser beam;
an overhead camera positioned at a second end of the arm, the overhead camera configured to detect the laser beam;
a wearable head unit comprising a laser for generating the laser beam; and
a controller configured to receive data from the overhead camera and the first camera indicative of a position of the laser beam, and in response to the received data, actuate the actuatable arm to adjust the position of the light.

2. The surgical lighting system of claim 1, wherein the actuating the actuatable arm comprises:
adjusting the position of the light to track a line of sight of the laser if the received data from the first camera indicates that the laser beam is within a field of view of the first camera;
initiating a search procedure if the data received from the first camera indicates that the laser beam is not within a field of view of the first camera, but the data received from the overhead camera indicates that the laser beam is within a field of view of the overhead camera; and
maintaining the position of the light if the data received from the first camera indicates that the laser beam is not within a field of view of the first camera, and the data received from the overhead camera indicates that the laser beam is not within a field of view of the overhead camera.

3. The surgical lighting system of claim 1, further comprising a LiDAR system for detecting heights of objects in an area within physical reach of the actuatable arm.

4. The surgical lighting system of claim 3, wherein data from the LiDAR system is provided to a microprocessor to determine an operation zone based on the heights of objects in the area, a working zone based on a furthest extension of the surgical lighting system, and a safety zone between the operation zone and the working zone.

5. The surgical lighting system of claim 4, wherein the safety zone is configured to prevent collisions between the surgical lighting system and any objects or people in a surgical suite.

6. The surgical lighting system of claim 1, wherein the actuatable arm comprises:
a plurality of booms; and
one or more motors, each motor of the one or more motors coupling adjacent booms of the plurality of booms.

7. The surgical lighting system of claim 1, further comprising:
safety glasses configured to identify a user's point of focus.

8. The surgical lighting system of claim 7, wherein the safety glasses comprise a laser module for generating the laser beam.

9. The surgical lighting system of claim 1, further comprising a remote control to control the surgical lighting system in remote-control mode.

10. The surgical lighting system of claim 9, further comprising a user interface for providing visual feedback during remote-control mode.

11. The surgical lighting system of claim 10, wherein the user interface depicts images from both the first camera and the overhead camera.

12. An autonomous surgical lighting system, comprising:
an actuatable arm;
a light source coupled to the actuatable arm and configured to generate a light;
a plurality of cameras; and
a controller, comprising:
a memory comprising executable instructions; and
a processor in data communication with the memory and configured to execute the executable instructions to:
receive image data from the plurality of cameras;
determine a location of a user's point of focus in a surgical field based on the image data; and
actuate the actuatable arm to adjust a position of the light to the location.

13. The autonomous surgical lighting system of claim 12, wherein the light source is a light head subsystem comprising a plurality of light emitting diode (LED) arrays.

14. The autonomous surgical lighting system of claim 13, wherein the light head subsystem is brightly colored.

15. The autonomous surgical lighting system of claim 13, wherein the light head subsystem is mounted to the actuatable arm through a motorized joint.

16. A system for lighting a surgical field, comprising:
a memory comprising executable instructions that when executed by a processor perform a method, comprising:
receiving information from a light head camera indicative of a user's point of focus;
determining whether the user's point of focus is within a surgical field; and
adjusting a light head to track the user's point of focus by adjusting one or more motorized joints along an actuatable arm until the user's point of focus is within an adjustable tolerance to the center of the image.

17. The system of claim 16, wherein the method further comprises:
receiving information from an overhead camera indicative of a user's point of focus; and
determining whether the user's point of focus is within a surgical field.

18. The system of claim 16, wherein the system can be autonomously controlled or remote controlled.

19. The system of claim 16, wherein the user's point of focus may be determined using colored gloves or colored tools.

20. The system of claim 16, wherein the user's point of focus may be determined from a laser point projected from safety glasses worn by the user.
